(19) United States
(12) Patent Application Publication
(10) Pub. No.: US 2013/0310436 A1
(43) Pub. Date: Nov. 21, 2013

(54) METHODS FOR PREDICTING RESPONSE TO BETA-BLOCKER THERAPY IN NON-ISCHEMIC HEART FAILURE PATIENTS

(71) Applicant: The Regents of the University of Colorado, a body corporate, Denver, CO (US)

(72) Inventors: Brian Lowes, Omaha, NE (US); Michael R. Bristow, Englewood, CO (US); David Kao, Denver, CO (US)

(21) Appl. No.: 13/897,203

(22) Filed: May 17, 2013

Related U.S. Application Data
(60) Provisional application No. 61/648,650, filed on May 18, 2012.

(51) Int. Cl. G06F 19/00; A61K 31/404

(52) U.S. Cl. CPC G06F 19/345; A61K 31/404
USPC 514/415; 702/19

(57) ABSTRACT

The present invention provides methods and apparatuses for predicting mortality and determining the likelihood of success of β-blocker therapy in a patient who has suffered non-ischemic heart failure as well as providing probability of short-term and/or long term survival rate of a non-ischemic heart failure patient. In addition, the present invention provides a method for determining a treatment procedure for a non-ischemic heart failure patient.

### CROSS-REFERENCE TO RELATED APPLICATIONS

This application claims the priority benefit of U.S. Provisional Application No. 61/648,650, filed May 18, 2012, which is incorporated herein by reference in its entirety.

### FIELD OF THE INVENTION

The present invention relates to methods and apparatuses for determining or predicting response to a β-blocker therapy in a patient who has suffered non-ischemic heart failure. The present invention also relates to determining probability of short-term and/or long term survival rate of a non-ischemic heart failure patient as well as a method for determining a treatment procedure for a non-ischemic heart failure patient.

### BACKGROUND OF THE INVENTION

It is believed that heart failure with reduced left ventricular ejection fraction (HFREF) develops from complex interactions between genetic factors and accumulated cardiac insults. Like all heart failure patients, HFREF patients are heterogenous with respect to etiology, prognosis, and response to therapy, and ability to identify patients likely to respond to medical therapy remains limited. In some cases, HFREF etiology directs therapy that increases the likelihood of clinical improvement. Forms of HFREF considered ‘reversible’ are often characterized by a single identifiable etiology amenable to targeted intervention. However, currently there is no reliable method for predicting treatment response in non-ischemic HFREF patients where a reversible etiology cannot be identified.

Today, left ventricular ejection fraction (LVEF) is primarily used to determine timing of medical and invasive therapies. However, normalization of LVEF in some patients with non-ischemic HFREF on medical therapy in the absence of an obvious reversible etiology suggests that there may be uncharacterized reversible phenotypes.

Therefore, there is a need for a method for determining a treatment procedure for a non-ischemic heart failure patient. There is also a need for a method for predicting a β-blocker therapy response in a non-ischemic heart failure patient.

### SUMMARY OF THE INVENTION

Some aspects of the invention are based on a discovery by the present inventors of patterns of disease defined by groups of clinical features that occur within the non-ischemic HF population. Using bioinformatics, the present inventors have identified subtypes and stages of non-ischemic HF patients. In some embodiments, subtypes and stages of non-ischemic HF patients were identified and correlated with their implications for clinical course and β-blocker response. In particular, the present inventors have discovered that high-dimensional clinical phenotyping (HDCP) and latent class analysis (LCA) provided a novel method of identifying subtypes and stages of non-ischemic HF that can be used in determining personalized prognosis and treatment.

Other aspects of the present invention are based at least in part on a retrospective analysis of data from the β-blocker Evaluation of Survival Trial (BEST) using a high-throughput clinical phenotyping method applied to clinical data available at the time of randomization followed by latent class analysis to identify prevalent subtypes and stages of non-ischemic dilated cardiomyopathy which the present inventors have then correlated with clinical endpoints. The present inventors have compared the results of this analysis with survival predictions made using the Seattle Heart Failure Model (SHFM) and assessed the utility of SHFM in predicting response to a β-blocker, such as bucindolol, carvedilol, propanolol, etc. Other exemplary β-blockers include, but are not limited to, alprenolol, carteolol, labetalol, nadolol, oxprenolol, penbutolol, pindolol, sotalol, timolol, eucommia bark, acebutolol, atenolol, betaxolol, bisoprolol, celiprolol, esmolol, metoprolol, nebivolol, and butaxamine. It should be appreciated, that the scope of the present invention also includes other β-blockers that may be approved by the Food and Drug Administration (FDA).

In other embodiments, methods of the invention are based on a calculated SHFM Score, and evaluation of a β-blocker (e.g., bucindolol) treatment, HF subtype, HF stage and

SHFM using univariate and multivariate analyses for prediction of all-cause and one-year mortality and improvement in ejection fraction (EF).

The present inventors have identified 6 HF subtypes (also referred to herein as LCM A subtypes) and 5 HF stages (also referred to herein as LCM B subtypes). The present inventors have found that HF subtype, HF stage, and SHFM were associated with significant variation in all-cause mortality, one-year mortality and improvement in EF. On multivariate analysis, HF subtype and SHFM remained significantly associated with all outcomes. Area under the receiver operating characteristic (ROC) curve improved with addition of HF subtype and HF stage information to SHFM for both outcomes. Predictive performance and the added value of subtype and stage of HF to SHFM in predicting LVEF response were similar by both validation methods.

Still other aspects of the invention are based on the present inventors' use of HDCP to identified HF subtypes and HF stages that have a significant correlation for prognosis including likelihood of positive response to a β-blocker.

One particular aspect of the invention provides a method for predicting response to a β-blocker therapy in a non-ischemic heart failure patient. Such a method comprises:
identifying a non-ischemic heart failure patient; and determining said non-ischemic heart failure patient's heart failure subtype (HF subtype), heart failure stage (HF stage), or a combination thereof.
   HF subtype 2, 3, 5 or 6, or HF stage 1 or 2, or a combination thereof is an indication that said non-ischemic heart failure patient will likely respond positively to a β-blocker treatment. The term “respond positively to a β-blocker treatment” means the non-ischemic HF patient's LVEF improves, e.g., increases by at least 5 LVEF units with a final LVEF of ≧35% in the absence of death or need for heart transplantation, when placed on a β-blocker treatment.

In some embodiments, said step of determining HF subtype comprises obtaining a plurality of clinical information from the non-ischemic HF patient and determining the HF subtype using the values listed in Table 1. Exemplary clinical information used in determining HF subtype include, but are not limited to, age when the clinical information is obtained, age of non-ischemic HF onset, gender, race, body mass index (BMI), presence of diabetes mellitus (DM), blood pressure, total cholesterol, triglycerides, estimated creatinine clearance (CrCl), hematocrit, atrial fibrillation, left bundle branch block (LBBB), history of pacemaker placement, personal history of sudden cardiac death (SCD), and the presence or absence of mitral valve disease and aortic valve disease. In some instances at least six, typically at least twelve, and ideally all of these clinical information are used to determine HF subtype of said non-ischemic HF patient. It should be appreciated that in general, the higher the number clinical information used in determining HF subtype, better the correlation of HF patient's subtype.

In other embodiments, said step of determining HF stage of said non-ischemic HF patient comprises obtaining a plurality of clinical information comprising: age when the plurality of clinical information is obtained, age of non-ischemic HF onset, left-ventricular ejection fraction (LVEF), right ventricular ejection fraction (RVEF), QRS complex duration, resting heart rate, systolic blood pressure (SBP), diastolic blood pressure (DBP), jugular venous distention (JVD), blood urea nitrogen (BUN), alanine aminotransferase (ALT), serum sodium level, body mass index (BMI), estimated creatinine clearance (CrCl), and hematocrit. In some instances at least six, typically at least twelve, and ideally all of these clinical information are used to determine HF stage of said non-ischemic HF patient. Again, it should be appreciated that in general, the higher the number clinical information used in determining HF stage, better the correlation of HF patient's stage.

Determining HF subtype or HF stage typically comprises determining probability of each HF subtype or HF stage of said non-ischemic heart failure patient using the variables provided in Tables 1 and 2. After calculating the probability of each HF subtype and/or HF stage, the highest HF subtype and/or HF stage probability is assigned or deemed to belong to said non-ischemic heart failure patient.

Another aspect of the invention provides a method for treating a non-ischemic heart failure patient. In some instances, this method is used to determine whether to place the non-ischemic HF patient on a β-blocker therapy or some other treatment course. The method of this aspect of the invention includes:
identifying a non-ischemic heart failure patient; determining said non-ischemic heart failure patient's heart failure subtype (HF subtype), heart failure stage (HF stage), or a combination thereof; and
treating said non-ischemic heart failure patient with a β-blocker therapy when said non-ischemic heart failure patient's HF subtype is 2, 3, 5 or 6, or when said non-ischemic heart failure patient's HF stage is 1 or 2, or a combination thereof.

Yet another aspect of the invention provides a method for determining a treatment procedure for a non-ischemic heart failure patient. In some embodiments, this method is used to determine whether the non-ischemic HF patient can be treated with β-blocker therapy or whether the patient will require a more extensive procedure such as requiring a cardiac resynchronization or heart replacement therapy. The method of this aspect of the invention typically includes:
identifying a non-ischemic heart failure patient;
determining said non-ischemic heart failure patient's heart failure subtype (HF subtype), heart failure stage (HF stage), or a combination thereof; and
placing said non-ischemic heart failure patient on a β-blocker therapy when said non-ischemic heart failure patient's HF subtype is 2, 3, 5 or 6, or HF stage is 1 or 2, or a combination thereof.

In some embodiments, when said non-ischemic heart failure patient's HF subtype is not 2, 3, 5 or 6 (i.e., is 1 or 4), or HF stage is not 1 or 2 (i.e., is 3, 4 or 5), then the patient is fitted with a biventricular pacemaker or further evaluated for heart replacement therapy such as transplantation or a left ventricular assist device. In some embodiments, when a widened QRS complex defined as a QRS duration >120 ms is present, the patient is fitted with a biventricular pacemaker, i.e., a biventricular pacemaker is placed in the patient.

Another aspect of the invention provides an apparatus for determining a non-ischemic HF patient's HF subtype, HF stage or a combination thereof. Such an apparatus typically includes:
a database comprising probability coefficient (or probability values) of Table 1, a database comprising probability coefficient (or probability values) of Table 2, or a combination thereof;
an input device for entering a plurality of clinical information of the non-ischemic HF patient or for extracting a patient's relevant information from an electronic medical record;
a central processing unit (CPU) for determining the patient's HF subtype, HF stage, or a combination thereof using the probability coefficient and the patient's plurality of clinical information; and
an output device for communicating the patient's HF subtype, HF stage, or a combination thereof to an operator.

The input device can be remotely connected to the database. In some embodiments, the database is centrally located so that it can be accessed remotely and simultaneously by many different operators. In fact, such database can be connected via the internet so that anyone with a proper access code and the internet connection can access the data base.

The input device can be a wireless input device. In this manner, a portable input device can be used. Such a wireless input device also allows an operator to input the clinical information directly while conversing with the patient.

### BRIEF DESCRIPTION OF THE DRAWINGS

FIG. 1 is a Table showing outcomes according to subtype.

FIG. 2 is a Table of Cox proportional hazards ratio, cumulative mortality for individual and combined models.

FIG. 3 is a Table of C-indices for predicting outcomes, BEST and validation sets (BEST, Last observation, MOCHA).

### DETAILED DESCRIPTION OF THE INVENTION

Some aspects of the invention provide methods and apparatuses for predicting likelihood of mortality in a non-ischemic HF patient, and/or determining the likelihood of β-blocker treatment success in a non-ischemic HF patient.

There are a variety of different clinical features among non-ischemic HF patients and that defining subtypes of such patients provides insight to underlying pathophysiology that can predict clinical course and be amenable to targeted therapy. The present inventors have found that classification of patients with respect to these subtypes allow personalized prediction of outcomes and likelihood of success of a β-blocker treatment. By analyzing data from a β-blocker evaluation of survival trial using high-throughput clinical phenotyping followed by latent class analysis to identify prevalent types and stages of non-ischemic dilated cardiomyopathy, the present inventors have discovered algorithms that can be used to predict likelihood of mortality of a given non-ischemic HF patient, and/or the likelihood of success of a β-blocker treatment for that patient.

The constitutions, profiles, or clinical conditions of the non-ischemic HF patient that were found to influence the HF subtype and stage include, but are not limited to, age at randomization (i.e., current age), age at diagnosis (i.e., age at first non-ischemic HF occurrence), gender, race, body mass index (BMI), LVEF, right ventricular ejection fraction (RVEF), QRS complex duration, resting heart rate, systolic blood pressure (SBP), diastolic blood pressure (DBP), pulse pressure, defined as SBP—DBP, hematocrit, jugular venous distention (JVD), blood urea nitrogen (BUN), alanine aminotransferase (ALT), serum sodium level, estimated creatinine clearance (CrCl), presence of diabetes mellitus (DM), hypertension (HTN), hyperlipidemia (HLD), hypertriglyceridemia (TRG), atrial fibrillation (AF), or left bundle branch block (LBBB), and history of pacemaker placement, SCD, mitral valve disease, and/or aortic valve disease. As can be seen in Tables 1 and 2, only a subset of these clinical conditions may be sufficient for determining HF subtype and HF stage. In some embodiment, at least six, typically at least 10, often at least 12, and more often at least 16 clinical conditions are used to determine the patient's HF subtype. In addition, as can be seen in Table 1, each HF subtype has an associated coefficient (e.g., age at onset of HF between 45-60 is associated with probability of 0.376 of membership in Type 1, 0.404 for Type 2, etc.). Typically, a patient's HF subtype is determined as follows:
First, the probability or odds of each HF type for a particular patient is determined by multiplying all of its coefficients for appropriate clinical conditions. This is done for all six HF types.
Second, the sum of HF Type probability is determined by adding the probability of HF types 1-6 for that patient.
And finally, the probability for a particular HF type is determined by dividing the probability of a particular HF type (from the first step) with the sum of HF type probability (from the second step).
   The patient is assigned a HF subtype based on the highest HF type probability obtained from the third step.

Similarly, in some embodiments, a patient's HF stage is determined using at least six, typically at least ten, often at least twelve, and more often at least fourteen clinical conditions. Furthermore, as with HF subtype determination, as can be seen in Table 2, each HF stage has associated coefficient, e.g., 0.229 for stage 1, 0.341 for stage 2, etc. Typically, the patient's HF stage is determined in a similar manner as determining the patient's HF subtype outlined above, but using coefficients in Table 2 rather than those of Table 1.

Other aspects of the invention provide devices and/or apparatuses that determine a patient's HF subtype and/or HF stage. Such apparatuses typically utilize the algorithms disclosed herein and often include an input device for entering the patient's relevant clinical condition information or for extracting a patient's relevant information from an electronic medical record, a database that stores the appropriate coefficients related to determining the patient's HF subtype and/or HF stage, and an output device that displays or communicates the patient's HF subtype and/or HF stage to a medical or an appropriate personnel. It should be appreciated that as one gathers more information, the coefficients in Table 1 and 2 can be updated to reflect additional data analysis. Accordingly, while the initial algorithms utilize coefficients provided herein, the scope of the invention is not limited to such coefficients. In fact, as more data is gathered and analyzed, it is expected that at least some of the coefficients in Tables 1 and 2 will change. Thus, methods and apparatuses of the invention include analyzing additional data as more patients are added to the study and using the coefficients derived therefrom.

In some particular embodiments, criteria (e.g., clinical conditions) were encoded and applied using a novel high-throughput phenotyping method developed by the present inventors within a MySQL server environment (Oracle Corporation, Redwood Shores, Calif.).

Apparatuses of the invention also includes a central processing unit (CPU) or a similar microprocessor that can rapidly calculate each of patient's HF subtype and/or HF stage probability as well as determine the patient's HF subtype and/or HF stage. The input device can be connected to the CPU via a cable or wire, or it can be operatively connected wirelessly, e.g., via BlueTooth®, infrared, Wi-Fi, etc., or any other wireless communication system that are known to one skilled in the art or are developed after disclosure of the present Application. Similarly, the output device can also be connected to the CPU via a cable, wire or wirelessly. The database can be centrally located (e.g., via a local area network, internet, intranet, or a similar networking system known to one skilled in the art or are developed after the disclosure of the present Application) such that it can be accessed by a plurality of users, or it can be incorporated into an individual apparatus for portability.

The term “HF subtype (or LCM A subtype) of a patient” refers to a highest probability of HF subtype calculated using values listed on Table 1. The term “HF stage (or LCM B subtype) of a patient” refers to a highest probability of HF stage calculated using values listed on Table 2. It should be noted that the values on Tables 1 and 2 were derived by using latent class analysis of 1121 patients with non-ischemic HFREF from the β-blocker evaluation of survival trial. Thus, as the sample size gets larger, the values listed in Tables 1 and 2 may change. Accordingly, it should be appreciated that the methods of the invention are not limited to the exact values listed in Tables 1 and 2. The scope of the invention includes modified values of Tables 1 and 2 as the number of sample size in latent class analysis increases.

The following example illustrates how one can determine LCM A subtype (i.e., HF subtype) for a non-ischemic HF patient. The following is the hypothetical non-ischemic patient's clinical information: White woman with HF onset at age 51, BMI 28 kg/m², creatinine clearance of 55 ml/min*1.73 m², hyperlipidemia with a total cholesterol of 210 mg/dL, hypertriglyceridemia with serum triglycerides=240 mg/dL, hypertension with a blood pressure of 150/90 mm Hg, left bundle branch block, and a hematocrit of 37%. The following are Bayesian partial probability calculations using coefficients (i.e., probability values or conditional probability) from the Table 1:
A=0.186 (population share)*0.376 (age=51)*0.177 (female)*0.381 (white)*0.193 (BMI=28)*0.739 (no diabetes)*0.643 (BP 150/90)*0.194 (cholesterol=210 mg/dL)*0.177 (triglycerides=240 mg/dL)*0.504 (creatinine clearance=55)* 0.556 (hematocrit=37%)*0.747 (no atrial fibrillation)*0.227 (left bundle branch block present)*0.924 (no pacemaker)*0.971 (no mitral valve disease)* 0.960 (no aortic valve disease)*0.968 (no history of sudden cardiac death)=3.83502*10⁻⁷
A2 (category coefficients in same order as for A1)=0.144*0.404*0.551*0.464* 0.326*0.305*0.583*0.164*0.217*0.511*0.376*0.891*0.199*1*1*1 *0.955=9.98265*10⁻⁷
A3=6.18331*10 ⁻⁵
A4=1.41343*10⁻⁷
A5=1.12669*10⁻⁷
A6=0

Sum of partial probabilities for LCM A₁₋₆=3.83*10⁻⁷+9.98*10⁻⁷+6.18*10⁻⁵+1.41*10⁻⁷+1.13*10⁻⁷+0=6.35*10⁻⁵. Thus, the final probability of class membership for each LCM A is:
A1: 3.83*10⁻⁷/6.35*10⁻⁵=0.0060
A2: 9.98*10⁻⁷/6.35*10⁻⁵=0.016
A3: 6.183*10⁻⁵/6.35*10⁻⁵=0.974 ->patient classified as A3
A4: 1.41*10⁻⁷/6.35*10⁻⁵=0.0022
A5: 1.13*10⁻⁷/6.35*10⁻⁵=0.0018
A6: 0/6.35*10⁻⁵=0
   Because A3 has the highest probability for latent class model A, this patient's HF subclass is determined to be A3.

TABLE 1
Class conditional probabilities for latent class model A

| | | A1 | A2 | A3 | A4 | A5 | A6 |
|---|---|---|---|---|---|---|---|
| | | 18.6% | 14.4% | 16.6% | 14.5% | 7.8% | 28.3% |
| | | (208) | (161) | (186) | (162) | (87) | (317) |
| Age of HF onset, years | <30 | 0.000 | 0.033 | 0.000 | 0.336 | 0.015 | 0.021 |
| | 30-45 | 0.154 | 0.251 | 0.182 | 0.540 | 0.118 | 0.299 |
| | 45-60 | 0.376 | 0.404 | 0.519 | 0.124 | 0.376 | 0.495 |
| | >60 | 0.470 | 0.312 | 0.300 | 0.000 | 0.491 | 0.184 |
| Gender | Male | 0.823 | 0.449 | 0.342 | 0.502 | 0.782 | 1.000 |
| | Female | 0.177 | 0.551 | 0.658 | 0.498 | 0.218 | 0.000 |
| Race | White, non-Hispanic | 0.381 | 0.464 | 0.942 | 0.343 | 0.854 | 0.677 |
| | Black, non-Hispanic | 0.529 | 0.442 | 0.025 | 0.545 | 0.080 | 0.242 |
| | Hispanic | 0.072 | 0.077 | 0.034 | 0.113 | 0.021 | 0.053 |
| | Asian/Pacific Islander | 0.018 | 0.006 | 0.000 | 0.000 | 0.024 | 0.010 |
| | American Indian | 0.000 | 0.011 | 0.000 | 0.000 | 0.000 | 0.014 |
| | Other | 0.000 | 0.000 | 0.000 | 0.000 | 0.022 | 0.003 |
| Body Mass Index, kg/m² | <18.5 | 0.078 | 0.000 | 0.028 | 0.000 | 0.030 | 0.000 |
| | 18.5-25 | 0.624 | 0.179 | 0.319 | 0.258 | 0.496 | 0.119 |
| | 25-30 | 0.193 | 0.326 | 0.316 | 0.238 | 0.370 | 0.368 |
| | >30 | 0.105 | 0.495 | 0.338 | 0.505 | 0.104 | 0.514 |
| Diabetes Mellitus | None | 0.739 | 0.305 | 0.846 | 0.906 | 0.930 | 0.687 |
| | Present | 0.203 | 0.481 | 0.096 | 0.088 | 0.047 | 0.227 |
| | Present with end-organ damage | 0.058 | 0.214 | 0.058 | 0.006 | 0.023 | 0.086 |
| Hypertension | None | 0.158 | 0.000 | 0.145 | 0.173 | 0.282 | 0.011 |
| | Borderline | 0.114 | 0.064 | 0.411 | 0.231 | 0.343 | 0.178 |
| | Present | 0.634 | 0.583 | 0.418 | 0.428 | 0.295 | 0.589 |
| | Severe | 0.093 | 0.353 | 0.026 | 0.168 | 0.080 | 0.221 |
| Total cholesterol, mg/dL | <200 | 0.642 | 0.069 | 0.128 | 0.576 | 0.420 | 0.262 |
| | 200-240 | 0.194 | 0.164 | 0.272 | 0.265 | 0.295 | 0.224 |
| | >240 | 0.164 | 0.766 | 0.600 | 0.159 | 0.285 | 0.514 |
| Triglycerides, mg/dL | <150 | 0.883 | 0.061 | 0.109 | 0.450 | 0.399 | 0.050 |
| | 150-250 | 0.117 | 0.217 | 0.335 | 0.364 | 0.321 | 0.386 |
| | >250 | 0.000 | 0.690 | 0.556 | 0.186 | 0.280 | 0.564 |
| Creat. Cl., ml/min * 1.73 m² | >90 | 0.030 | 0.046 | 0.100 | 0.332 | 0.058 | 0.139 |
| | 60-90 | 0.358 | 0.273 | 0.431 | 0.517 | 0.277 | 0.531 |
| | 30-60 | 0.504 | 0.511 | 0.436 | 0.152 | 0.581 | 0.301 |
| | 15-30 | 0.097 | 0.143 | 0.033 | 0.000 | 0.083 | 0.029 |
| | <15 | 0.011 | 0.026 | 0.000 | 0.000 | 0.000 | 0.000 |
| Hematocrit, % | >40 | 0.049 | 0.000 | 0.000 | 0.000 | 0.056 | 0.126 |
| | 30-40 | 0.556 | 0.376 | 0.473 | 0.488 | 0.580 | 0.874 |
| | 20-30 | 0.366 | 0.608 | 0.527 | 0.492 | 0.364 | 0.000 |
| | <20 | 0.028 | 0.016 | 0.000 | 0.020 | 0.000 | 0.000 |
| Atrial fibrillation | Yes | 0.253 | 0.109 | 0.083 | 0.058 | 0.806 | 0.221 |
| | No | 0.747 | 0.891 | 0.917 | 0.942 | 0.194 | 0.779 |
| Left bundle branch block | Yes | 0.227 | 0.199 | 0.670 | 0.081 | 0.107 | 0.158 |
| | No | 0.773 | 0.801 | 0.330 | 0.919 | 0.893 | 0.842 |
| Pacemaker | Yes | 0.076 | 0.000 | 0.006 | 0.043 | 0.379 | 0.036 |
| | No | 0.924 | 1.000 | 0.994 | 0.957 | 0.621 | 0.964 |
| Mitral valve disease | Yes | 0.029 | 0.000 | 0.032 | 0.014 | 0.430 | 0.021 |
| | No | 0.971 | 1.000 | 0.968 | 0.986 | 0.570 | 0.979 |
| Aortic valve disease | Yes | 0.040 | 0.000 | 0.014 | 0.000 | 0.201 | 0.011 |
| | No | 0.960 | 1.000 | 0.986 | 1.000 | 0.799 | 0.989 |
| History of sudden cardiac death | Yes | 0.032 | 0.045 | 0.054 | 0.032 | 0.133 | 0.021 |
| | No | 0.968 | 0.955 | 0.946 | 0.968 | 0.867 | 0.979 |

Similarly, determination process is used to determine the patient's LCM B subtype using the Table 2.

TABLE 2
Class conditional probabilities for latent class model B

| | | B1 | B2 | B3 | B4 | B5 |
|---|---|---|---|---|---|---|
| | | 22.9% | 34.1% | 22.4% | 11.6% | 9.1% |
| Population share | | (247) | (368) | (242) | (125) | (98) |
| Age, years | <30 | 0.000 | 0.035 | 0.000 | 0.208 | 0.057 |
| | 30-45 | 0.009 | 0.293 | 0.073 | 0.507 | 0.197 |
| | 45-60 | 0.269 | 0.487 | 0.425 | 0.267 | 0.385 |
| | >60 | 0.727 | 0.185 | 0.502 | 0.018 | 0.361 |
| LVEF, % | >55 | 0.004 | 0.000 | 0.000 | 0.000 | 0.000 |
| | 45-55 | 0.000 | 0.000 | 0.000 | 0.000 | 0.000 |
| | 35-45 | 0.008 | 0.003 | 0.000 | 0.000 | 0.000 |
| | 25-35 | 0.584 | 0.493 | 0.211 | 0.351 | 0.139 |
| | <25 | 0.405 | 0.504 | 0.789 | 0.649 | 0.861 |
| RVEF % | >55 | 0.209 | 0.071 | 0.065 | 0.059 | 0.000 |
| | 45-55 | 0.208 | 0.185 | 0.095 | 0.129 | 0.080 |
| | 35-45 | 0.275 | 0.296 | 0.188 | 0.244 | 0.247 |
| | 25-35 | 0.193 | 0.267 | 0.315 | 0.227 | 0.151 |
| | <25 | 0.115 | 0.181 | 0.338 | 0.342 | 0.522 |
| QRS, msec | <120 | 0.389 | 0.756 | 0.453 | 0.735 | 0.574 |
| | 120-150 | 0.235 | 0.066 | 0.189 | 0.082 | 0.214 |
| | >150 | 0.376 | 0.179 | 0.358 | 0.183 | 0.213 |
| Heart rate, bpm | <60 | 0.131 | 0.051 | 0.061 | 0.042 | 0.054 |
| | 60-80 | 0.541 | 0.370 | 0.367 | 0.233 | 0.190 |
| | 80-100 | 0.310 | 0.419 | 0.492 | 0.469 | 0.499 |
| | 100-120 | 0.019 | 0.147 | 0.074 | 0.197 | 0.227 |
| | >120 | 0.000 | 0.013 | 0.007 | 0.059 | 0.031 |
| Systolic blood pressure, mmHg | >120 | 0.698 | 0.664 | 0.079 | 0.000 | 0.229 |
| | 110-120 | 0.203 | 0.267 | 0.158 | 0.064 | 0.108 |
| | 100-110 | 0.086 | 0.066 | 0.299 | 0.401 | 0.191 |
| | 90-110 | 0.013 | 0.003 | 0.306 | 0.421 | 0.256 |
| | <90 | 0.000 | 0.000 | 0.158 | 0.115 | 0.215 |
| Pulse pressure, mmHG | >40 | 0.909 | 0.670 | 0.103 | 0.132 | 0.267 |
| | 25-40 | 0.091 | 0.319 | 0.773 | 0.730 | 0.511 |
| | <25 | 0.000 | 0.012 | 0.123 | 0.137 | 0.222 |
| Jugular venous distension | Not present | 0.603 | 0.632 | 0.527 | 0.555 | 0.292 |
| | Base of neck | 0.281 | 0.235 | 0.293 | 0.237 | 0.230 |
| | Halfway up | 0.088 | 0.108 | 0.139 | 0.151 | 0.319 |
| | Angle of mandible | 0.028 | 0.025 | 0.041 | 0.057 | 0.159 |
| Blood Urea Nitrogen, mg/dL | <10 | 0.018 | 0.174 | 0.025 | 0.163 | 0.000 |
| | 10-25 | 0.679 | 0.777 | 0.679 | 0.794 | 0.082 |
| | 25-40 | 0.192 | 0.048 | 0.250 | 0.011 | 0.388 |
| | 40-55 | 0.071 | 0.000 | 0.046 | 0.031 | 0.217 |
| | >55 | 0.040 | 0.000 | 0.000 | 0.313 | 0.779 |
| Alanine aminotransferase, U/L | <25 | 0.779 | 0.464 | 0.598 | 0.377 | 0.535 |
| | 25-50 | 0.206 | 0.442 | 0.320 | 0.510 | 0.265 |
| | 50-75 | 0.015 | 0.072 | 0.073 | 0.080 | 0.131 |
| | >75 | 0.000 | 0.022 | 0.010 | 0.034 | 0.068 |
| Serum sodium, mEq/L | >140 | 0.378 | 0.346 | 0.301 | 0.172 | 0.138 |
| | 130-140 | 0.610 | 0.650 | 0.690 | 0.802 | 0.799 |
| | <130 | 0.012 | 0.004 | 0.009 | 0.026 | 0.062 |
| Body Mass Index, kg/m² | <18.5 | 0.034 | 0.005 | 0.053 | 0.005 | 0.000 |
| | 18.5-25 | 0.403 | 0.153 | 0.425 | 0.288 | 0.365 |
| | 25-30 | 0.386 | 0.249 | 0.332 | 0.313 | 0.197 |
| | >30 | 0.177 | 0.594 | 0.191 | 0.394 | 0.438 |
| Creat. Clearance, ml/min * 1.73 m² | >90 | 0.021 | 0.204 | 0.000 | 0.383 | 0.000 |
| | 60-90 | 0.278 | 0.611 | 0.365 | 0.549 | 0.050 |
| | 30-60 | 0.576 | 0.180 | 0.635 | 0.061 | 0.059 |
| | 15-30 | 0.110 | 0.005 | 0.000 | 0.000 | 0.342 |
| | <15 | 0.015 | 0.000 | 0.000 | 0.008 | 0.018 |
| Hematocrit, % | >40 | 0.011 | 0.032 | 0.064 | 0.068 | 0.101 |
| | 30-40 | 0.457 | 0.710 | 0.625 | 0.531 | 0.443 |
| | 20-30 | 0.521 | 0.258 | 0.307 | 0.370 | 0.422 |
| | <20 | 0.010 | 0.000 | 0.005 | 0.031 | 0.035 |

As stated above, some aspects of the invention are based on the discovery by the present inventors that subtypes of non-ischemic HFREF exist that can be differentiated by collection of clinical features or information that reflect underlying pathophysiology. These subtypes have variable clinical courses and responses to treatment, and identification of these subtypes can also provide insight into mechanisms of HFREF and facilitate personalized prediction or determination of outcomes and treatment response.

Traditional outcomes-driven analyses are limited in the number of clinical features that can be evaluated due to the number of potential interactions between features contributing to the development and progression of HFREF. The present inventors' use of latent class analysis provided identification of groups of individuals within a population that share similar patterns of categorical variables such as symptoms or comorbid conditions. Others have used latent class analysis, for example, for exploring heart failure, characterization, and validation of diseases subtypes as well as for risk stratification and prediction of treatment response. Latent class analysis has also been used to establish diagnostic standards for complex disease syndromes, and use of latent class analysis has been proposed as a method of dealing with large numbers of complex interactions and multiple comparisons in determining likelihood of response to interventions.

Briefly, latent class analysis hypothesizes the existence of unobserved classes within a population that explain patterns of association between variables and uses maximum-likelihood estimation to divide the population into subgroups by calculating a probability of subgroup membership for each symptom or comorbidity. An individual's subgroup membership thus depends on the presence or absence of many different characteristics in a given model.

When the population in question has a shared disease, the results are data-driven definitions of disease subtypes where each subtype is characterized by a distinct combination of clinical features. Many clinical variables can thereby be incorporated into an analytic model while preserving statistical power for outcomes analysis by identifying the most prevalent combinations of variables upon which to focus.

The present inventors have used complex phenotype descriptions of patients in combination with latent class analysis to identify subtypes of non-ischemic HFREF that have different prognoses and likelihoods of treatment response. Some aspects of the invention are based on a retrospective analysis of data from the β-blocker Evaluation of Survival Trial (BEST) that generated high-dimensional phenotype descriptions of subjects using clinical data available at the time of randomization. Latent class analysis was then used to identify prevalent subtypes of HFREF, and the effect of a β-blocker, such as bucindolol, treatment on mortality and LVEF response was determined for each subtype. The present inventors also compared the performance of methods of the invention with the Seattle Heart Failure Model (SHFM) in predicting patient mortality and LVEF improvement with a β-blocker and estimated the incremental value of combining models. Models were validated by estimating unbiased area-under-the-curve c-indices within the BEST population and by applying latent class and SHFM models to an independent set of patients enrolled in the Multicenter Oral Carvedilol Heart Failure Assessment (MOCHA) Trial.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### Examples

Methods: A total of 1121 patients with non-ischemic HFREF from the β-blocker Evaluation of Survival Trial were categorized according to 27 clinical features. See Tables 1 and 2 for the clinical features or information used. Latent class analysis was used to generate two latent class models, LCM A and B, Tables 3 and 4, respectively, to identify HFREF subtypes. LCM A consisted of features associated with HF pathogenesis, whereas LCM B consisted of markers of HF progression and severity. The Seattle Heart Failure Model (SHFM) Score was also calculated for all patients. Mortality, improvement in left ventricular ejection fraction (LVEF) defined as an increase in LVEF ≧5% and a final LVEF of 35% after 12 months, and effect of bucindolol on both outcomes were compared across HFREF subtypes. Performance of models that included a combination of LCM subtypes and SHFM scores towards predicting mortality and LVEF response was estimated and subsequently validated using leave-one-out cross-validation and data from the Multicenter Oral Carvedilol Heart Failure Assessment (MOCHA) Trial.

Results: A total of 6 subtypes were identified using LCM A (Table 3) and 5 subtypes using LCM B (Table 4). Several subtypes resembled familiar clinical phenotypes. Prognosis, improvement in LVEF, and the effect of bucindolol treatment differed significantly between subtypes. Prediction improved with addition of both latent class models to SHFM for both 1-year mortality and LVEF response outcomes.

Conclusions: The combination of high-dimensional phenotyping and latent class analysis were used to determine or identify subtypes of HFREF. These subtypes were found to be useful analytical tools in determining prognosis and response to specific therapies. These subtypes might also be used to determine mechanisms of disease as well developing personalized treatment plan(s) for a non-ischemic HF patient.

Methods: All patients had New York Heart Association (NYHA) class III or IV HFREF (LVEF ≦35%) and were randomized in a double-blind fashion to either bucindolol (a β-blocker) or placebo. Patients were considered ischemic if they had ≧70% obstruction in a major epicardial coronary artery by angiography or evidence of prior myocardial infarction and excluded from this analysis. The primary endpoint was cumulative all-cause mortality. Secondary endpoints were all-cause mortality at one year and LVEF response defined as improvement in LVEF ≧5% with a final LVEF of ≧35% as measured using multi-gated acquisition scan (MUGA). All patients had an LVEF ≦35%, were mostly NYHA class II or III and had stable HF symptoms for 1 month prior to enrollment. They were randomized to placebo, low (6.25 mg bid), medium (12.5 mg bid), or high-dose (25 mg bid) carvedilol. Death and LVEF improvement as measured by MUGA were secondary endpoints in the original MOCHA analysis. Mortality data was only available up to one year of follow-up in MOCHA.

Identification and definition of latent classes: Patients were scored according to 27 clinical features, i.e., clinical information (Tables 3 and 4). Criteria were encoded and applied in a MySQL server environment (Oracle Corporation, Redwood Shores, Calif.). Patient clinical information were analyzed collectively using latent class analysis applied to two sets of clinical variables that were designated as Latent Class Models (LCM) A and B (Tables 3 and 4). LCM A and B differed only in the clinical variables included in each model. LCM A included variables that describe a patient's non-cardiac characteristics that can contribute to the pathogenesis of HFREF including age, gender, race, body mass index, and presence of comorbidities such as diabetes, atrial fibrillation, or valvular disease.

TABLE 3
Features of HFREF Latent Class Model A subtypes

| | | A1 | A2 | A3 | A4 | A5 | A6 | All |
|---|---|---|---|---|---|---|---|---|
| HF Subset | | (18.6) | (14.4) | (16.6) | (14.5) | (7.8) | (28.3) | n = 1121 |
| Age of HF onset, years | <30 | 0.0 | 3.7 | 0.0 | 35.8 | 1.1 | 1.6 | 6.2 |
| | 30-45 | 13.9 | 25.5 | 17.7 | 56.2 | 11.5 | 29.7 | 26.6 |
| | 45-60 | 38.9 | 38.5 | 52.2 | 8.0 | 37.9 | 50.2 | 39.7 |
| | >60 | 47.1 | 32.3 | 30.1 | 0.0 | 49.4 | 18.6 | 27.5 |
| Male | | 83.7 | 39.1 | 30.1 | 47.5 | 79.3 | 100.0 | 67.4 |
| Race | White, non-Hispanic | 36.1 | 42.2 | 96.2 | 34.0 | 87.4 | 68.8 | 59.9 |
| | Black, non-Hispanic | 54.8 | 47.2 | 1.1 | 55.6 | 5.7 | 23.0 | 32.1 |
| | Hispanic | 7.7 | 8.7 | 2.7 | 10.5 | 2.3 | 5.4 | 6.3 |
| | Asian/Pacific Islander | 1.4 | 0.6 | 0.0 | 0.0 | 2.3 | 1.3 | 0.9 |
| | American Indian | 0.0 | 1.2 | 0.0 | 0.0 | 0.0 | 1.3 | 0.5 |
| | Other | 0.0 | 0.0 | 0.0 | 0.0 | 2.3 | 0.3 | 0.3 |
| Body Mass Index, kg/m² | <18.5 | 7.7 | 0.0 | 2.7 | 0.0 | 2.3 | 0.0 | 2.1 |
| | 18.5-25 | 63.9 | 17.4 | 33.3 | 27.2 | 50.6 | 10.7 | 30.7 |
| | 25-30 | 19.2 | 31.7 | 31.2 | 23.5 | 37.9 | 36.9 | 30.1 |
| | >30 | 9.1 | 50.9 | 32.8 | 49.4 | 8.0 | 52.4 | 37.0 |
| Diabetes Mellitus | None | 73.1 | 25.5 | 88.2 | 92.6 | 94.3 | 67.2 | 71.5 |
| | Present | 21.6 | 51.6 | 7.0 | 6.8 | 3.4 | 23.7 | 20.5 |
| | Present + end-organ damage | 5.3 | 23.0 | 4.8 | 0.6 | 2.3 | 9.1 | 7.9 |
| Blood pressure, mmHg | <120/80 | 15.9 | 0.0 | 14.0 | 19.1 | 31.0 | 0.6 | 10.6 |
| | 120-140/80-90 | 10.1 | 5.0 | 44.6 | 22.2 | 34.5 | 18.0 | 21.0 |
| | 140-160/90-100 | 65.4 | 57.8 | 39.8 | 41.4 | 27.6 | 59.0 | 51.8 |
| | >160/100 | 8.7 | 37.3 | 1.6 | 17.3 | 6.9 | 22.4 | 16.6 |
| Total cholesterol, mg/dL | <200 | 65.4 | 6.2 | 9.7 | 61.7 | 42.5 | 24.6 | 33.8 |
| | 200-240 | 18.8 | 15.5 | 28.5 | 26.5 | 29.9 | 22.4 | 22.9 |
| | >240 | 15.9 | 78.3 | 61.8 | 11.7 | 27.6 | 53.0 | 43.3 |
| Triglycerides, mg/dL | <150 | 91.7 | 8.9 | 10.9 | 48.4 | 40.2 | 2.3 | 30.2 |
| | 150-250 | 8.3 | 20.9 | 33.7 | 37.1 | 32.9 | 39.5 | 28.4 |
| | >250 | 0.0 | 70.3 | 55.4 | 14.5 | 26.8 | 58.2 | 38.7 |
| Creat. Cl., ml/min * 1.73 m² | >90 | 2.9 | 3.7 | 9.1 | 34.0 | 5.7 | 14.2 | 12.0 |
| | 60-90 | 37.0 | 24.2 | 41.9 | 52.5 | 25.3 | 52.7 | 41.7 |
| | 30-60 | 49.5 | 53.4 | 45.2 | 13.6 | 59.8 | 30.6 | 39.6 |
| | 15-30 | 9.6 | 15.5 | 3.8 | 0.0 | 9.2 | 2.5 | 6.1 |
| | <15 | 1.0 | 3.1 | 0.0 | 0.0 | 0.0 | 0.0 | 0.6 |
| Hematocrit, | >40 | 4.8 | 0.0 | 0.0 | 0.0 | 5.7 | 11.4 | 4.5 |
| | 30-40 | 57.2 | 28.6 | 45.7 | 45.7 | 58.6 | 88.6 | 58.5 |
| | 20-30 | 35.1 | 69.6 | 54.3 | 52.5 | 35.6 | 0.0 | 35.9 |
| | <20 | 2.9 | 1.9 | 0.0 | 1.9 | 0.0 | 0.0 | 1.1 |
| Atrial fibrillation | | 24.5 | 9.3 | 8.1 | 6.2 | 86.2 | 22.1 | 21.1 |
| Left bundle branch block | | 23.1 | 19.9 | 67.7 | 7.4 | 10.3 | 16.1 | 24.8 |
| Pacemaker | | 6.7 | 0.0 | 0.5 | 4.3 | 42.5 | 3.5 | 6.2 |
| Mitral valve disease | | 1.9 | 0.0 | 3.2 | 1.2 | 48.3 | 2.2 | 5.4 |
| Aortic valve disease | | 3.8 | 0.0 | 1.1 | 0.0 | 21.8 | 1.3 | 2.9 |
| History of sudden cardiac death | | 2.9 | 4.3 | 5.4 | 2.5 | 16.1 | 2.5 | 4.4 |

TABLE 4
Features of HFREF Latent Class Model B subtypes

| | | B1 | B2 | B3 | B4 | B5 | All subjects |
|---|---|---|---|---|---|---|---|
| Age, years | <30 | 0.0 | 3.2 | 0.0 | 22.1 | 7.0 | 4.3 |
| | 30-45 | 0.4 | 29.8 | 6.2 | 53.4 | 21.0 | 19.7 |
| | 45-60 | 24.9 | 49.9 | 43.4 | 23.7 | 38.0 | 38.6 |
| | >60 | 74.7 | 17.2 | 50.4 | 0.8 | 34.0 | 37.4 |
| LVEF, | >55 | 0.4 | 0.0 | 0.0 | 0.0 | 0.0 | 0.1 |
| | 45-55 | 0.0 | 0.0 | 0.0 | 0.0 | 0.0 | 0.0 |
| | 35-45 | 0.8 | 0.3 | 0.0 | 0.0 | 0.0 | 0.3 |
| | 25-35 | 60.1 | 48.3 | 19.8 | 35.1 | 15.0 | 39.9 |
| | <25 | 38.7 | 51.5 | 80.2 | 64.9 | 85.0 | 59.8 |
| RVEF, | >55 | 21.9 | 6.6 | 6.3 | 5.8 | 0.0 | 7.4 |
| | 45-55 | 20.4 | 19.0 | 9.5 | 11.7 | 8.3 | 12.3 |
| | 35-45 | 28.1 | 29.8 | 18.0 | 26.2 | 22.6 | 20.7 |
| | 25-35 | 18.4 | 26.9 | 32.0 | 22.3 | 13.1 | 19.9 |
| | <25 | 11.2 | 17.7 | 34.2 | 34.0 | 56.0 | 20.9 |
| QRS, msec | <120 | 36.0 | 77.3 | 45.0 | 76.3 | 56.0 | 58.5 |
| | 120-150 | 24.5 | 5.5 | 19.0 | 8.4 | 23.0 | 14.8 |
| | >150 | 39.5 | 17.2 | 36.0 | 15.3 | 21.0 | 26.7 |
| Heart rate, bpm | <60 | 13.4 | 5.3 | 5.0 | 4.6 | 6.0 | 7.0 |
| | 60-80 | 55.7 | 35.6 | 36.8 | 22.1 | 18.0 | 37.3 |
| | 80-100 | 29.6 | 42.2 | 50.8 | 46.6 | 49.0 | 42.5 |
| | 100-120 | 1.2 | 15.6 | 7.0 | 19.8 | 24.0 | 11.6 |
| | >120 | 0.0 | 1.3 | 0.4 | 6.9 | 3.0 | 1.6 |
| Systolic blood pressure | >120 | 70.8 | 63.6 | 6.2 | 0.0 | 22.0 | 41.7 |
| (mmHg) | 110-120 | 20.9 | 28.2 | 14.3 | 2.3 | 10.0 | 18.7 |
| | 100-110 | 7.1 | 5.3 | 31.4 | 44.3 | 20.0 | 17.6 |
| | 90-110 | 1.2 | 0.3 | 31.4 | 42.7 | 26.0 | 14.9 |
| | <90 | 0.0 | 0.0 | 16.7 | 10.7 | 22.0 | 7.0 |
| Pulse pressure, mmHg | >40 | 87.0 | 65.7 | 7.8 | 12.2 | 26.0 | 47.4 |
| | 25-40 | 6.3 | 30.9 | 79.5 | 74.0 | 49.0 | 43.2 |
| | <25 | 0.0 | 1.1 | 12.8 | 13.7 | 23.0 | 7.0 |
| Jugular venous | Not present | 60.1 | 63.3 | 51.2 | 58.0 | 26.0 | 55.8 |
| distension | Base of neck | 27.7 | 23.7 | 31.0 | 20.6 | 22.0 | 25.8 |
| | Halfway up | 8.7 | 10.6 | 14.0 | 15.3 | 35.0 | 13.6 |
| | Angle of mandible | 3.2 | 2.4 | 3.9 | 6.1 | 17.0 | 4.6 |
| Blood Urea Nitrogen, mg/dL | <10 | 1.6 | 17.4 | 2.3 | 16.8 | 0.0 | 8.7 |
| | 10-25 | 67.2 | 77.8 | 67.4 | 79.4 | 3.0 | 66.5 |
| | 25-40 | 19.8 | 4.7 | 25.6 | 0.8 | 38.0 | 15.4 |
| | 40-55 | 7.5 | 0.0 | 4.7 | 3.1 | 23.0 | 5.2 |
| | >55 | 4.0 | 0.0 | 0.0 | 0.0 | 35.0 | 4.0 |
| Alanine aminotransferase, U/L | <25 | 79.4 | 45.1 | 60.9 | 37.4 | 53.0 | 56.0 |
| | 25-50 | 19.4 | 44.9 | 31.0 | 53.4 | 25.0 | 35.1 |
| | 50-75 | 1.2 | 7.4 | 7.0 | 8.4 | 14.0 | 6.6 |
| | >75 | 0.0 | 2.4 | 0.8 | 3.1 | 8.0 | 2.1 |
| Serum sodium, mEq/L | >140 | 38.9 | 34.7 | 29.6 | 16.9 | 9.1 | 29.5 |
| | 130-140 | 59.8 | 65.0 | 69.6 | 80.0 | 83.8 | 67.3 |
| | <130 | 1.3 | 0.3 | 0.8 | 3.1 | 7.1 | 1.5 |
| Body Mass Index, kg/m² | <18.5 | 3.6 | 0.5 | 4.3 | 0.8 | 0.0 | 2.1 |
| | 18.5-25 | 41.1 | 14.5 | 43.8 | 29.0 | 35.0 | 30.7 |
| | 25-30 | 39.5 | 24.0 | 32.9 | 32.8 | 18.0 | 30.1 |
| | >30 | 15.8 | 60.9 | 18.6 | 37.4 | 47.0 | 37.0 |
| Creat. Clearance | >90 | 1.2 | 20.8 | 0.0 | 39.7 | 0.0 | 12.0 |
| (ml/min * 1.73 m²) | 60-90 | 26.5 | 61.5 | 34.9 | 56.5 | 4.0 | 41.7 |
| | 30-60 | 59.7 | 17.2 | 65.1 | 3.1 | 56.0 | 39.6 |
| | 15-30 | 11.1 | 0.5 | 0.0 | 0.0 | 38.0 | 6.1 |
| | <15 | 1.6 | 0.0 | 0.0 | 0.8 | 2.0 | 0.6 |
| Hematocrit, | >40 | 1.2 | 2.9 | 6.6 | 6.9 | 11.0 | 4.5 |
| | 30-40 | 43.1 | 72.6 | 63.6 | 49.6 | 43.0 | 58.5 |
| | 20-30 | 54.5 | 24.5 | 29.5 | 40.5 | 42.0 | 35.9 |
| | <20 | 1.2 | 0.0 | 0.4 | 3.1 | 4.0 | 1.1 |

LCM B included variables that generally describe cardiac function, progression, and severity of HFREF including right- and left-ventricular function, hemodynamic parameters such as heart rate and blood pressure, end-organ function such as estimated creatinine clearance, and signs of venous congestion such as jugular venous distension and alanine aminotransferase levels. In total, three variables were included in both models: body mass index, creatinine clearance, and hematocrit. All 3 variables have been implicated in the pathogenesis of HFREF and can also be markers of severity of HFREF. They were included in both models to illustrate that the variable implications of clinical features in different contexts may be represented using this approach. Two sets of related variables were also included: age of HF onset (LCM A) vs. chronologic age (LCM B) and presence of hypertension (LCM A) vs. presence of hypotension (LCM B). Age of HF onset, a static value, may be relevant to the HFREF etiology, while chronologic age may be related to HF progression. Similarly, presence of hypertension (LCM A) is related to HF etiology while hypotension (LCM B) is a marker of advanced HF.

Latent class analysis was performed using the poLCA function in the R statistical package. The optimal clinical profiles according to the variables in each latent class model were derived in the form of subtype-conditional probabilities for each variable for a range of 2-10 subtypes. Error statistics were calculated for each model iteratively to determine the optimum number of latent classes. The number of latent classes corresponding to the first local x² minimum following the first minimum of the Bayesian information criterion was selected, and the corresponding model was used for all subsequent analyses. The most likely LCM A and LCM B subtype were determined for each patient in a Bayesian fashion (See above), and descriptive statistics were compiled. SHFM Score and corresponding predicted mortality at one year were calculated for all patients both with and without the SHFM β-blocker coefficient. The SHFM Score including the β-blocker coefficient was used to assess overall performance of SHFM Score in the BEST population, and the SHFM Score excluding the β-blocker coefficient was used for all analyses investigating the treatment effect of a β-blocker. All multivariate predictors in the SHFM were available in the BEST trial with the exception of percent lymphocytes, and a value of 25% was imputed for all patients based on the validation sets for SHFM.

Association between latent class models and outcomes: Cox proportional-hazards models and the log-rank test were used to examine the associations between latent classes and cumulative all-cause mortality according to the intention-to-treat principle. These models were fit using the coxph and survfit functions from the survival library in the R statistical package. Logistic regression models were used for the one-year mortality and LVEF response outcomes. Interactions between latent classes and the treatment groups were used to estimate the response to treatment within each subtype. For survival models, an interaction with time was included for those variables that did not meet the proportional hazards assumption.

Multivariate models comprised of all possible combinations of LCM A, B, and SHFM Score were generated to identify those that provide the best discrimination between outcome variables. Cox proportional hazards models and the log-rank test were used to study discrimination of all-cause mortality, whereas logistic regression was used to study discrimination of one-year mortality and LVEF response. All models included treatment group as a covariate, and c-indices were calculated for model comparison. Improvement in risk prediction with the addition of LCM A and B to SHFM Score according to logistic regression was assessed by comparing c-indices with the x² test and by calculation of net reclassification improvement measures (NRI). Logistic regression, NRI, and ROC calculations were performed using SAS version 9.2 software. (SAS Institute Inc., Cary, N.C., 2008)

Validation of multivariate models: Performance of the Cox and logistic regression models in predicting all-cause mortality, one-year mortality and LVEF response was further validated by estimating unbiased c-indices for both outcomes using leave-one-out cross-validation in the BEST dataset and by applying the estimates from the predictive models calculated on the BEST population to nonischemic HFREF patients from the independent MOCHA population.

Results. Patient characteristics: In all, 1121/2708 patients enrolled in BEST were identified as nonischemic and included in all subsequent analyses. A total of 6 LCM A and 5 LCM B subtypes were identified. Distributions of clinical variables for all subjects according to subtype are shown in Tables 3 and 4, respectively. The subtype-conditional probabilities for each explanatory variable used to calculate an individual's LCM A and LCM B subtype are given in Tables 1 and 2, respectively.

Latent Class Model A (Table 3): LCM A subtypes were characterized by distinct collections of clinical features that frequently resembled known HFREF syndromes. Subtype A1 was characterized by advanced age of onset, non-Caucasian race, male gender, HTN, mild-moderate renal insufficiency, and elevated rates of atrial fibrillation (24.5%). Subtype A2 was characterized by middle age of onset, female gender, moderate renal insufficiency, anemia, high body mass index, and very high rates of diabetes mellitus (74.6%), hypertension (95.0%), hyperlipidemia (93.8%), and hypertriglyceridemia (91.1%). Subtype A3 was characterized by middle age of onset, female gender, Caucasian race, hyperlipidemia, hypertriglyceridemia, anemia, and the presence of left bundle branch block (LBBB). Subtype A4 was characterized by young age of onset, non-Caucasian race, obesity, anemia, and lower rates of traditional cardiac risk factors such as hyperlipidemia, hypertriglyceridemia, and diabetes mellitus. Subtype A5 was characterized by advanced age of onset, Caucasian race, atrial fibrillation (86.2%), mitral valve disease (48.3%), aortic valve disease (21.8%), history of pacemaker placement (42.5%), and a significantly higher rate of prior sudden cardiac death (16.1%). This subtype had the smallest number of subjects (7.8%), whereas subtype A6 was the largest with 28.3% of subjects. Subtype A6 was characterized by middle age of onset, Caucasian race, male gender (100%), high body mass index, hypertension, hyperlipidemia, and hypertriglyceridemia with less associated diabetes mellitus (32.8%) than was seen in Subtype A2.

Latent Class Model B (Table 4): Subjects were fairly evenly divided among LCM B subtypes with subtypes B3 and B5 having slightly smaller percentages of 11.7% and 8.9%, respectively. Subtypes B1-B3 were characterized by preserved systolic blood pressure, pulse pressure, RVEF, and renal function with few signs of volume overload such as jugular venous distention and elevated serum alanine aminotransferase. Ventricular function declined first followed by worsening of hemodynamic parameters and finally by signs of venous congestion. In subtypes B4 and B5, right ventricular ejection fraction, systolic and pulse pressure were lower, heart rate was higher, renal function and hyponatremia were worse, and jugular venous distension, blood urea nitrogen, and serum alanine aminotransferase were higher. Examination of features such as age and body mass index suggest that while many of the clinical features are markers of heart failure severity, LCM B subtypes may not represent a continuous progression of illness shared among all HFREF patients in this study.

Association with outcomes: In LCM A, subtype A1 had the highest event rates with 43.8% cumulative all-cause mortality, 18.3% one-year mortality, and an LVEF response rate of only 14.4%, whereas subtypes A2 and A6 had some much lower event rates with cumulative all-cause morality of 16.8% and 18.9%, respectively, and LVEF response rates of 31.1% and 36.0%, respectively. A much larger range in cumulative mortality rates were observed for the LCM B subtypes; subtype B5 had the highest overall mortality rate at 54.0% (62.2% for placebo -treated patients), while subtype B1 had the lowest at 15.3% (17.5% for placebo-treated patients). These ranks were consistent across one-year mortality and lack of LVEF response.

The predicted one-year mortality for all patients using SHFM was 11.0% compared with an observed one-year mortality of 9.6%. The hazard ratio (HR) and corresponding confidence interval (CI) for cumulative all-cause mortality associated with each unit increase in SHFM Score including the β-blocker coefficient was 2.92 (95% CI 2.40-3.54, p <0.0001). The c-index for SHFM Score including the β-blocker coefficient was 0.76 (95% CI 0.66-0.85), which was comparable to all SHFM validation sets (range 0.60-0.81). The likelihood of LVEF response decreased significantly with each unit increase in SHFM Score (OR 0.29, 95% CI 0.22-0.38), and SHFM performed well in predicting LVEF response (c-index=0. 68).

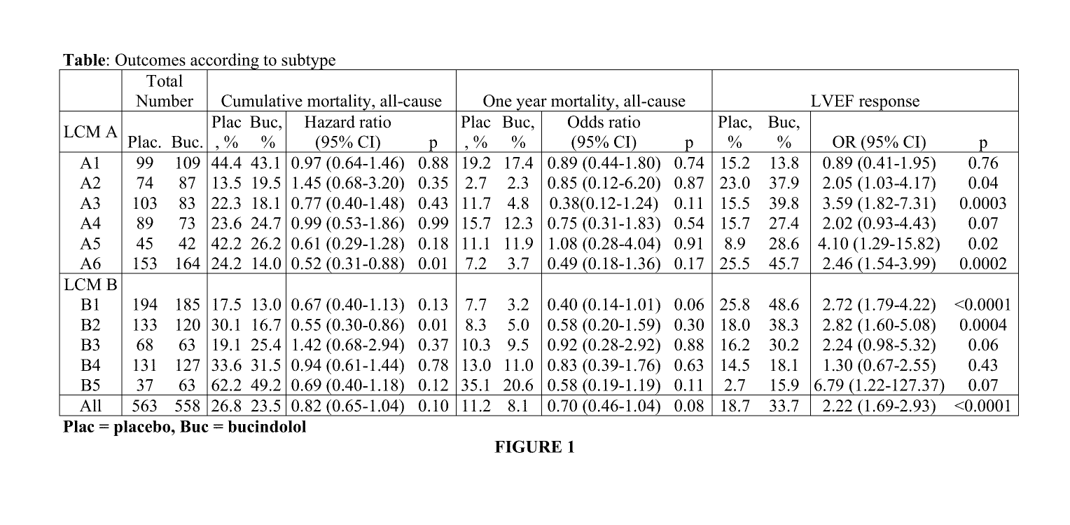

Differences in treatment ejects between latent classes: A total of 151/563 patients (26.8%) in the placebo group and 131/558 (23.5%) patients in the bucindolol group died (HR 0.82, 95% CI 0.65-1.04, p=0.1). Response to treatment was evaluated for all three outcomes within each subtype (Table in FIG. 1). Response to bucindolol as measured by cumulative survival varied significantly in both LCM A and B models. Subtype Al showed no reduction in cumulative all-cause mortality associated with bucindolol, whereas subtype A6 showed an absolute and relative risk reduction of 10.2% and 42% respectively (p=0.01). In LCM B, only subtype B2 showed significant improvement in mortality associated with bucindolol (p=0.01). There was a time-varying effect of treatment in LCM A2 and A4. Therefore, the HRs presented in Table 3 are average HRs over the observed death times. To further assess the effect of treatment within each LCM subtype at a single clinically meaningful time-point, the model using one-year mortality was also characterized fully.

The effect of bucindolol on one-year mortality trended towards benefit for those in the A6 and B2 classes, but did not reach statistical significance for either class. There was a marginally significant difference for A3 at 12 months, but this difference disappeared at subsequent follow-up. Bucindolol was associated with a significant increase in likelihood of LVEF response in subtypes A2, A3, A5, and A6 and ranged from no effect in subtype A1 to a 156% relative and 24.3% absolute increase in subtype A3. The likelihood of improvement in LVEF increased comparably across all B subtypes both in relative and absolute terms but only reached statistical significance in subtypes B1 and B2.

Combined models: Multivariate survival and logistic regression models were then constructed to determine whether LCM A and B classification added predictive information to each other and to the SHFM. Multivariate Cox hazard ratios for cumulative mortality are shown in Table 4. Consistent with the descriptive event rates presented earlier, subtype A1 had the highest cumulative mortality even after adjusting for LCM B and SHFM Score, and subtypes A2 and A6 had survival rates 50-70% better than those in A1. Significant time interactions were observed for subtypes A2 and A4. The HRs comparing the risk of subtype A2 with Al increased over time, with HR estimates from the full model at 1, 2, and 3 years of 0.32 (95% CI 0.19-0.52), 0.51 (95% CI 0.31-0.82) and 0.67 (95% CI 0.34-1.14), respectively. In contrast, the HRs comparing the risk of subtype A4 decreased over time with a HR at 1, 2, and 3 years were 0.69 (95% CI 0.44-1.08), 0.55 (95% CI 0.33-0.94), and 0.49 (95% CI 0.27-0.89), respectively. When LCM B and SHFM Score were combined, the risk for subjects in subtype B5 remained a significantly different from subjects in B1 (HR 2.12, 95% CI 1.35-3.34). When combined with SHFM Score, all subtypes except for A5 had a lower mortality compared to subtype A1. Both LCM A and B remained significant predictors of mortality after adjusting for risk associated with treatment and SHFM Score (p<0.01).

Multivariate logistic regression was performed (data not shown), and LCM A and B were highly significant for predicting both one-year mortality and LVEF response for both models (p<0.01). LCM A remained highly significant in predicting both mortality and LVEF response when combined with SHFM Score. LCM B remained significant in predicting LVEF response in combination with SHFM Score, but did not remain significant in predicting mortality. Membership in subtype B5 remained an independent predictor of mortality with a hazard ratio of 2.23 (95% CI 1.02-4.85) relative to subtype B1. When all three factors were included, LCM A and SHFM Score and subtype B5 remained significant predictors of one-year mortality. LCM A, B, and SHFM Score were all multivariate predictors of LVEF response.

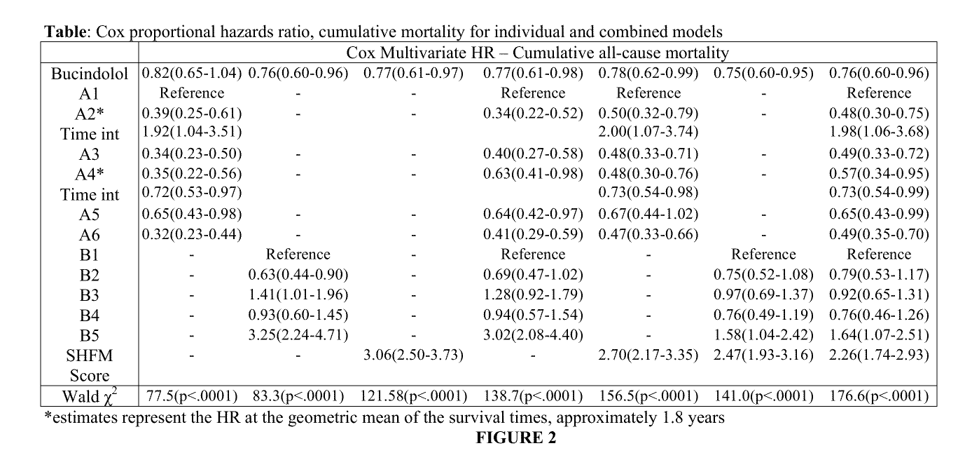

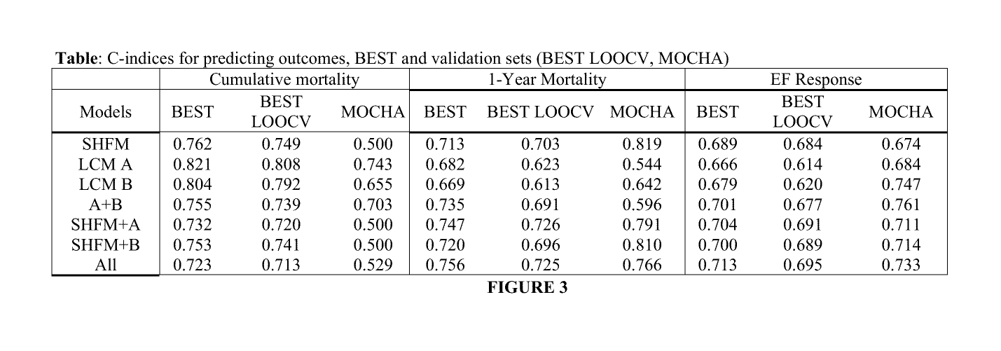

Model comparisons: Comparisons of all 7 combinations of predictors displayed in the Table shown in FIG. 2 were made for the three outcome measures (cumulative mortality, one-year mortality and LVEF response) using c-indices (Table in FIG. 3). The model which included LCM A alone appeared to have the best predictive ability for cumulative mortality overall. Models with LCM A, B and their combination performed at least as well as SHFM in predicting cumulative mortality, but any model that included the SHFM appeared to reduce performance according to the proportional hazards model. In contrast, the addition of LCM A and B to SHFM Score and bucindolol treatment improved prediction of one-year mortality according to logistic regression. The c-index for predicting one-year mortality increased from 0.71 to 0.75 with the addition of LCM A (p=0.02) and to 0.76 with the addition of LCM A and B (p<0.01). The NRI also showed a significant improvement in prediction of one-year mortality with the addition of LCM A and LCM A +B to SHFM and bucindolol treatment (p<0.01 for both). Adding both LCM A and B significantly increased the c-index for predicting LVEF response to 0.71 (p<0.01). The NRI showed statistically significant improvement with addition of LCM A and B individually as well as together (p<0.01 for all). NRI transition matrices are found in Table 5.

TABLE 5
Reclassification matrices when adding LCM A, LCM B and both LCM A and
B to SHFM + treatment group.

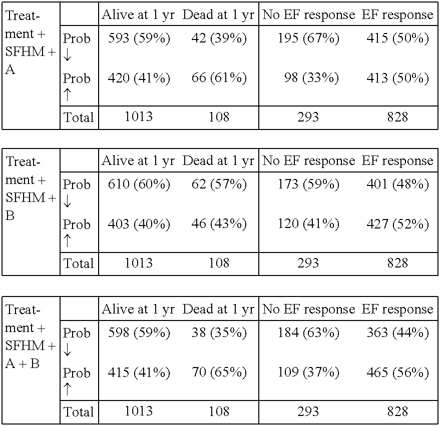

*Improved performance is implied by higher percentages in grey-shaded boxes and lower percentages in non-shaded boxes

Validation: Leave-one-out-cross-validation and external validation were then performed to verify association between latent class membership and outcomes as well as the added value of combining latent class models with the SHFM. As expected, c-indices for all models for all outcomes were slightly lower using leave-one-out cross-validation (FIG. 3). The added value of LCM A alone and the combination of LCM A and B to SHFM and treatment group in predicting one-year mortality and LVEF response were redemonstrated, as was the decrease in performance of predicting cumulative all-cause mortality with combined LCM and SHFM models. The LCM A and B subgroup definitions were then used to classify the 166 non-ischemic HFREF patients enrolled in MOCHA. One-year mortality and LVEF response were similar in non-ischemic patients enrolled in MOCHA and BEST. Survival models that included SHFM did not perform well in MOCHA, though the low number of deaths (6) and a much shorter follow-up time (median=6 months) likely contributed to the low predictive ability for cumulative mortality using the proportional hazards model. In contrast, models that included SHFM had somewhat better predictive ability for the one-year mortality outcome. Finally, the c-index for predicting LVEF response in the validation dataset increased using SHFM Score and bucindolol alone (c=0.67) with the addition of LCM A (c=0.71), B (c=0.71), and both A and B (c=0.73).

Discussion: Using the combination of high-dimensional clinical phenotyping and latent class analysis, the present inventors have identified a number of HFREF subtypes with distinct clinical profiles that demonstrate significant variation in prognosis as measured by all-cause mortality and response to a β-blocker as measured by reduction in mortality and increased likelihood of LVEF response. Several of the LCM A subtypes resemble previously described nonischemic HFREF phenotypes, while LCM B subtypes model HF progression and severity. The latent class models, particularly LCM A, remained significantly associated with certain outcomes after combining them with the SHFM, indicating that the information in the latent class models is different from the information in the SHFM Score. Taken together, these results show that methods of the invention can be used to identify patients with potentially ‘reversible’ HFREF as well as those more likely to benefit from a β-blocker therapy.

Insight into mechanisms of disease and treatment response: Regression models often provide only limited insight into the interactions between clinical processes in a given individual or the importance of a specific process in different contexts. The features of the subtypes identified in the present analysis indicate methods of the invention can be used to identify cohorts of patients who share underlying pathophysiology and disease prognosis. Defining clinical features of LCM A subtypes, corresponding outcomes, and treatment response are summarized in Table below.

Table of key features of Latent Class Model A,
mortality trends, and response to bucindolol

| | | | Bucindolol response | |
|---|---|---|---|---|
| LCM A | | Baseline | Improved | Improved |
| Subtype | Key clinical features | mortality | survival | EF |
| A1 - | Old age of onset | [symbol] | − | − |
| Hypertensive | Non-Caucasian | | | |
| cardiomyopathy | Male | | | |
| of the elderly | Atrial fibrillation | | | |
| phenotype | Hypertension | | | |
| | Normal BMI | | | |
| | Moderate Renal Insufficiency | | | |
| A2 - Insulin | Middle age of onset | [symbol] | − | + |
| resistant | Female | | | |
| cardimyopathy | Obesity | | | |
| (female) | Diabetes mellitus | | | |
| phenotype | Hypertension | | | |
| | Hyperlipidemia/hypertriglyceridemia | | | |
| | Anemia | | | |
| A3 - Left | Middle age of onset | [symbol] | − | + |
| bundle branch | Female | | | |
| block | Caucasian | | | |
| cardiomyopathy | Left bundle branch block | | | |
| phenotype | Hyperlipidemia/hypertriglyceridemia | | | |
| A4 - Idiopathic | Young age of onset | [symbol] | − | − |
| dilated | Non-Caucasian | | | |
| cardiomyopathy | Obesity | | | |
| phenotype | Anemia | | | |
| A5 - Valvular | Old age of onset | [symbol] | − | + |
| cardiomyopathy | Atrial fibrillation | | | |
| of the elderly | Mitral or aortic valve disease | | | |
| phenotype | H/o pacemaker placement | | | |
| | Normal BMI | | | |
| | H/o sudden death | | | |
| A6 - Insulin- | Middle age of onset | [symbol] | + | + |
| resistant | Male | | | |
| cardiomyopathy | Caucasian | | | |
| (male) | Obesity | | | |
| phenotype | Hypertension | | | |
| | Hyperlipidemia/hypertriglyceridemia | | | |
| | Diabetes mellitus (less than A2) | | | |

Subtypes A1 and A5, both characterized by older age, are notable for high rates of hypertension and valvular disease respectively, which have been identified as two major risk factors for non-ischemic HFREF in the elderly. Without being bound by any theory, it is believed that the development of HFREF in the setting of these risk factors is in part due to accumulated DNA damage and telomere attrition, which affect cellular function and apoptosis. These two subtypes also had the highest event rates in LCM A, as might be expected in cardiomyopathy of the elderly. Subtypes A2 and A6 are distinguished by obesity, hypertension, hyperlipidemia, hypertriglyceridemia and diabetes mellitus, which are all characteristics of the metabolic syndrome, and these subtypes may therefore represent variants of insulin-resistant cardiomyopathy. Of note, subtype A2 had the lowest cumulative and one-year mortality and showed no significant reduction in mortality with bucindolol, while subtype A6, the largest LCM A subtype, was the only LCM A subtype to show a decrease in all-cause mortality. Subtype A3 patients are characterized primarily by the presence of LBBB, a known risk factor for HFREF. LBBB may affect neurohormonal activation and promote myocyte hypertrophy and LV dilation as consequences of ventricular dysynchrony. While some features of the LCM A subtypes are familiar, novel associations were observed such as the predominance of white women and the high prevalence of dyslipidemia in subtype A3, which was characterized primarily by LBBB. Such observations may provide new insight into the mechanisms involved in the pathogenesis of HFREF. In addition, subtypes such as A6 that demonstrated a favorable response to bucindolol even when the overall effect of bucindolol was not significant may be used to determine the specific role of a β-blocker in treatment of HFREF in those patients as well as the mechanism of treatment response.

Trends of key features of the LCM B subtypes are represented below:

Specifically, LCM B subtypes with worse prognoses were associated with first with worsening LV and RV ejection fraction followed by evidence of worsening cardiac output (higher heart rate, lower systolic and pulse pressure, and hyponatremia) and finally worsening evidence of volume overload (increasing jugular venous distension, blood urea nitrogen, and alanine aminotransferase). Each of these trends has been associated with poor prognosis in HFREF, and even in the analyses that have characterized several markers of severity simultaneously, there has been little quantitative insight gained into the patterns or order in which worsening prognostic signs may appear. In addition, treatment benefit with bucindolol for both all-cause mortality and LVEF response was confined to LCM B stages with lower baseline mortality, higher LV and RVEF, more favorable hemodynamics, and few signs of volume overload. Results of analyses with the SHFM, which uses similar variables (i.e. gender, 100/serum cholesterol, 100/LVEF, systolic blood pressure/10, age/10, 138-serum sodium, |16-serum hemoglobin), also indicated higher likelihood of LVEF response associated with lower SHFM scores, but the LCM A and B models provide concrete clinical profiles to associate with treatment response.

Identification of HFREF subtypes using latent class analysis: This analysis demonstrates the potential utility of combining high-dimensional clinical phenotyping and latent class analysis for identifying relevant subtypes of HFREF. It is difficult to determine multivariate odds ratios for all of the variables included in the latent class models presented here using a traditional regression model, as the number of possible interactions (26,542,080 and 432,000,000 for LCM A and LCM B, respectively) prevents calculation using realistic sample sizes. Latent class analysis provides a quantitative mechanism of reducing the number of comparisons by aggregating individuals with similar clinical profiles. Methods of the invention produced data-driven definitions of HFREF subtypes that integrate a large number of clinical features but are not dependent on any one feature for classification. Consequently, a feature like age may not have the same implications among all individuals. For example, subtype A4 is associated with worse outcomes than subtypes A2 or A6 despite younger age and lower burden of comorbid diseases. Clinical features may therefore be associated with a conditional probability for different outcomes depending on their context, capturing relevant interactions between comorbid conditions without direct calculation of all possible interactions. The added value of LCM A and B membership to SHFM for predicting survival despite sharing several common variables suggests that LCM A and B subtype may provide additional prognostic information to the SHFM Score. The variability in clinical outcomes observed between subtypes shows that this approach is useful in identifying patients with higher likelihood of HFREF reversibility in the absence of an obvious reversible etiology or conversely for identifying high risk patients for accelerated advanced HFREF therapy.

Implementation and sharing: Latent class analysis produces a formal mechanism for classifying any individual patient according to the subtypes presented in this analysis. While manual calculation is possible, an electronic implementation is more convenient. This can be accomplished using a stand-alone web-based application or by incorporation into an electronic health record system. Furthermore, simplified criteria can be developed for identifying LCM A and B types using methods such as classification and/or regression trees to simplify clinical application and validation in other data sets.

Conclusion: High-dimensional phenotyping combined with latent class analysis provide a method of identifying subtypes of non-ischemic HFREF patients who have shared pathophysiology with implications for prognosis and response to a β-blocker therapy. Significant reduction in all-cause mortality and increase in likelihood of LVEF response was associated with bucindolol treatment in specific groups identified using these classification methods. Identification of patients' HFREF subtype provides a means of personalizing clinical prognosis and estimating or determining likelihood of responding to medical treatment.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for predicting response to a β-blocker therapy in a non-ischemic heart failure patient, said method comprising:
identifying a non-ischemic heart failure patient; and
determining said non-ischemic heart failure patient's heart failure subtype (HF subtype), heart failure stage (HF stage), or a combination thereof;
wherein HF subtype 2, 3, 5 or 6, or HF stage 1 or 2, or a combination thereof is an indication that said non-ischemic heart failure patient will likely respond positively to a β-blocker treatment.

2. The method of claim 1, wherein said step of determining HF subtype from said non-ischemic heart failure patient comprises the step of obtaining a plurality of clinical information comprising: age when the plurality of clinical information is obtained, age of non-ischemic HF onset, gender, race, body mass index (BMI), presence of diabetes mellitus (DM), blood pressure, total cholesterol, triglycerides, estimated creatinine clearance (CrCl), hematocrit, atrial fibrillation, left bundle branch block (LBBB), history of pacemaker placement, personal history of sudden cardiac death (SCD), mitral valve disease, and aortic valve disease.

3. The method of claim 2, wherein said step of determining HF subtype comprises obtaining at least six of said clinical information from said non-ischemic heart failure patient.

4. The method of claim 2, wherein said step of determining HF subtype comprises obtaining at least twelve of said clinical information from said non-ischemic heart failure patient.

5. The method of claim 1, wherein said step of determining HF stage from said non-ischemic heart failure patient comprises the step of obtaining a plurality of clinical information comprising: age when the plurality of clinical information is obtained, age of non-ischemic HF onset, left-ventricular ejection fraction (LVEF), right ventricular ejection fraction (RVEF), QRS complex duration, resting heart rate, systolic blood pressure (SBP), diastolic blood pressure (DBP), jugular venous distention (JVD), blood urea nitrogen (BUN), alanine aminotransferase (ALT), serum sodium level, body mass index (BMI), estimated creatinine clearance (CrCl), and hematocrit.

6. The method of claim 5, wherein said step of determining HF stage comprises obtaining at least six of said clinical information from said non-ischemic heart failure patient.

7. The method of claim 5, wherein said step of determining HF stage comprises obtaining at least twelve of said clinical information from said non-ischemic heart failure patient.

8. The method of claim 1, wherein said step of determining HF subtype comprises determining probability of each HF subtype of said non-ischemic heart failure patient, wherein the highest HF subtype probability is assigned said non-ischemic heart failure patient's HF subtype.

9. The method of claim 1, wherein said step of determining HF stage comprises determining probability of each HF stage of said non-ischemic heart failure patient, wherein the highest HF stage probability is assigned said non-ischemic heart failure patient's HF stage.

10. A method for treating a non-ischemic heart failure patient, said method comprising:
identifying a non-ischemic heart failure patient;
determining said non-ischemic heart failure patient's heart failure subtype (HF subtype), heart failure stage (HF stage), or a combination thereof; and
treating said non-ischemic heart failure patient with a β-blocker therapy when said non-ischemic heart failure patient's HF subtype is 2, 3, 5 or 6, or when said non-ischemic heart failure patient's HF stage is 1 or 2, or a combination thereof.

11. A method for determining a treatment procedure for a non-ischemic heart failure patient, said method comprising:
identifying a non-ischemic heart failure patient;
determining said non-ischemic heart failure patient's heart failure subtype (HF subtype), heart failure stage (HF stage), or a combination thereof; and
placing said non-ischemic heart failure patient on a β-blocker therapy when said non-ischemic heart failure patient's HF subtype is 2, 3, 5 or 6, or HF stage is 1 or 2, or a combination thereof.

12. The method of claim 11, wherein when said non-ischemic heart failure patient's HF subtype is not 2, 3, 5 or 6, or HF stage is not 1 or 2, then placing a biventricular pacemaker in said non-ischemic heart failure patient or proceeding with evaluation of said non-ischemic heart failure patient for heart replacement therapy or a left ventricular assist device.

13. The method of claim 12, wherein said biventricular pacemaker is placed in said non-ischemic heart failure patient when a widened QRS complex is present.
